(19) United States
(12) Patent Application Publication
(10) Pub. No.: US 2020/0016758 A1
(43) Pub. Date: Jan. 16, 2020

(54) METHOD FOR CALIBRATING A MANIPULATOR OF A DIAGNOSTIC AND/OR THERAPEUTIC MANIPULATOR SYSTEM

(71) Applicant: KUKA Deutschland GmbH, Augsburg (DE)

(72) Inventors: Henrik Keller, Augsburg (DE); Philip Mewes, Nürnberg (DE)

(21) Appl. No.: 16/471,068

(22) PCT Filed: Dec. 15, 2017
(86) PCT No.: PCT/EP2017/001428

§ 371 Date: Jun. 19, 2019

(30) Foreign Application Priority Data
Dec. 20, 2016 (DE) 10 2016 225 613.0

(51) Int. Cl. B25J 9/16; A61B 34/30

(52) U.S. Cl. CPC B25J 9/1692; A61B 2017/00725; A61B 34/30

(57) ABSTRACT

A method for calibrating a manipulator of a diagnostic and/or therapeutic manipulator system, wherein the manipulator system includes at least one medical imaging device. The method includes at least: a) moving the manipulator to at least one target pose; b) capturing at least one image of at least a part of the manipulator and/or at least of a part of an end effector of the manipulator with the medical imaging device if the manipulator has moved to the target pose; c) determining the actual pose of the manipulator using the captured image; d) determining the deviation between the target pose and the actual pose of the manipulator; and e) calculating at least one calibration parameter on the basis of the determined deviation, and calibrating the manipulator.

### CROSS-REFERENCE TO RELATED APPLICATIONS

This application is a national phase application under 35 U.S.C. § 371 of International Patent Application No. PCT/EP2017/001428, filed Dec. 15, 2017 (pending), which claims the benefit of priority to German Patent Application No. DE 10 2016 225 613.0, filed Dec. 20, 2016, the disclosures of which are incorporated by reference herein in their entirety.

### TECHNICAL FIELD

The invention relates to a method for calibrating a manipulator of a diagnostic and/or therapeutic manipulator system as well as a control device equipped to carry out the method. The invention also relates to a manipulator system, comprising the control device equipped for carrying out the method.

### BACKGROUND

Diagnostic and/or therapeutic manipulator systems are known from the prior art and comprise at least one manipulator that can be used for diagnostic and/or therapeutic purposes. Manipulators are devices, which enable a physical interaction with the surroundings. Typical manipulators are industrial robots or jointed arm robots, comprising three or more freely programmable axes of movement (links), which can be guided automatically. These manipulators are used in various applications, either as stationary or mobile devices. In particular, they are equipped to guide end effectors or workpieces. End effectors may be various tools. In the field of diagnostic and/or therapeutic manipulator systems, end effectors may comprise, for example, surgical instruments, scalpels, needle holders for preparing surgical sutures, systems for inserting biopsy needles, trocars and the like. Additional examples of end effectors comprise endoscopes, staplers, etc.

Manipulators and jointed arm robots in particular are used in larger and larger numbers in the medical field. They may be used in automated manipulator systems, partially automated manipulator systems and teleoperated manipulator systems. In automated and partially automated manipulator systems, (partial) tasks are carried out independently by the manipulator. In teleoperated manipulator systems, the manipulator is controlled manually by a surgeon via suitable input devices. The manipulator converts the input commands of the input device into corresponding manipulator movements. The surgeon and the manipulator may be separated spatially in this process.

For example, partially automated manipulator systems are used in the field of tissue biopsy, to position a biopsy needle and orient it in such a way that the tissue to be biopsied is targeted reliably when the biopsy needle is advanced, i.e., when the skin is punctured. The needle may be advanced manually or by means of teleoperated systems. Other known applications relate to the placement of implants, in particular the placement of surgical screws.

The therapeutic and/or diagnostic applications are often monitored and/or planned by means of medical imaging devices. Thus, for example, monitoring is necessary when positioning surgical screws, to ensure that no nerves or any of the patient's other tissue structures are inadvertently damaged by the screw. Known imaging devices supply (two-dimensional or three-dimensional) image data of a patient's organs and structures. Imaging devices can be systematized according to how their images are produced, for example, by means of x-rays (e.g., x-ray devices, C arms, computer tomography), radionuclides (e.g., scintiscan, positron emission tomography, single-photon emission computer tomography), ultrasound (for example, sonography, color Doppler) and nuclear magnetic resonance (e.g., magnetic resonance tomography). The imaging device may also comprise optical sensors such as cameras, which create images based on visible light.

In comparison with tools that are guided strictly by hand, manipulator-guided tools (end effectors) have the advantage that they can be used repeatedly and positioned accurately without being dependent on human sources of error, such as a tremor, lack of concentration, fatigue and the like. To achieve a high accuracy with the manipulator and, in particular, the end effector, manipulators are typically calibrated and/or adjusted.

In determining the accuracy of a manipulator, a distinction is made between repeat accuracy and absolute accuracy. Absolute accuracy refers to the deviation between the actual pose of the end effector and the anticipated target pose of the end effector, wherein the poses are typically specified in relation to an external reference coordinate system. For example, the manipulator basic coordinate system is used as the basis. The absolute accuracy of a manipulator is usually determined by measuring the mean or maximum deviation between the target pose and the actual pose, which results during the approach to the target pose from various directions (multidirectionally). A pose comprises the position and the orientation of the end effector. Repeat accuracy indicates exactly how one manipulator is positioned in multiple approaches to a pose from the same direction and is to be evaluated as the deviation between the actual poses achieved. The repeat accuracy can be measured without knowledge of the exact position of the basic coordinate system because it is not necessary to use a target pose for comparison.

Various factors have a negative influence on both types of accuracy. In particular, axial zero positions and errors in length and angle between the individual links of the manipulator have negative effects on accuracy. Additional error sources comprise varying loads as well as wear and heating. When manipulators are used in diagnostic and/or therapeutic applications, the manipulator must have a high accuracy (in particular a high absolute accuracy) to minimize the risk to the patient or to enable specific applications, for example, microsurgery.

It is known that both the repeat accuracy and the absolute accuracy can be increased by calibration of the robot. Factors having a negative effect on accuracy are therefore measured and captured in kinematic models or correction matrices in order to be used as calibration parameters in operation of the manipulator. The increased accuracy achieved by means of calibration increases over time, for example, due to wear on gears, mechanical fatigue or extreme use scenarios (emergency stopping at a high velocity), thereby necessitating a renewed calibration.

Various measurement systems can be used for calibration of the manipulator. These comprise systems of laser interferometry, theodolites, measurement probes or laser triangulation devices. These measurement systems are characterized by a very high measurement accuracy but they are usually expensive and cannot readily be transported, so their use in the medical field, such as in an operating room, is made difficult or even impossible.

Calibration is necessary in particular when the calibration performed in the production of the manipulator, for example, is no longer valid due to transport of the manipulator to the site of use and/or new or altered parameters, e.g., geometric parameters, must be incorporated into the calibration because the manipulator is assembled at the site of use. In addition, the use of replacement parts or the occurrence of extreme use scenarios, such as emergency stops or the like, may necessitate renewed calibration. Consequently, calibration at the site of use is often unavoidable and is responsible for high costs because measurement systems must be brought to the site of use of the manipulator and put into operation there.

The object of the present invention is to at least partially eliminate the advantages described above and to make available a method for calibration of a manipulator of a diagnostic and/or therapeutic manipulator system, which is accurate and can be carried out easily at the site of use of the manipulator.

### SUMMARY

This object is achieved by a method for calibrating a manipulator, a control device, a manipulator system, or by a computer-readable medium as described herein.

In particular, this object is achieved by a method for calibrating a manipulator of a diagnostic and/or therapeutic manipulator system, wherein the manipulator system comprises at least one medical imaging device, and wherein the method comprises at least the following steps:

a) Approaching at least one target pose by means of the manipulator (10);
b) Capturing at least one image of at least one part of the manipulator and/or at least one part of an end effector of the manipulator by means of the medical imaging device when the manipulator has approached the target pose;
c) Determining the actual pose of the manipulator by means of the captured image;
d) Determining the deviation between the target pose and the actual pose of the manipulator;
e) Calculating at least one calibration parameter based on the deviation determined; and
f) Calibrating the manipulator.

The at least one target pose may be approached by the manipulator from various directions, to be able to calculate the most precise possible calibration parameters.

The image is captured by means of the medical imaging device in such a way that at least one part of the manipulator and/or of the end effector is captured in the image. This means that the target pose must either be selected, so that at least one part of the manipulator and/or one part of the end effector protrudes into the image capture range of the imaging device when the manipulator assumes the target pose, or so that the imaging device must be positioned and oriented accordingly in order to be able to capture the manipulator/end effector in the target pose.

The target pose is typically defined by the tool center point of the end effector and describes the position and orientation of the end effector in space. If redundant manipulators are used, i.e., manipulators typically having more than six degrees of freedom, then additional parameters may be necessary for unambiguous determination of the target pose. A plurality of parts of the manipulator or the entire manipulator may also be captured in capturing the image accordingly.

In particular, capturing at least one image by means of the medical imaging device is to be understood to mean that the medical imaging device is used for producing images as if the medical imaging device were creating images of a patient for diagnosis or treatment. However, this does not rule out that the medical imaging device will be operated for capturing the image in such a way that the actual pose of the manipulator can be recognized particularly well but a patient cannot be recognized well. For example, an MRI may be used to capture the image in a first mode, which captures the manipulator, the end effector or the marker particularly clearly. For capture of the patient, i.e., for the actual medical imaging, the MRI may then be operated in a second mode.

Furthermore, it does not matter that the captured image is actually transmitted in the form of a visual impression (e.g., as a photo or the like) to a user/operator or to a device carrying out the method. Instead, capture of the image comprises capture of image raw data suitable for being converted into images typical of the medical imaging device.

For example, if an ultrasonic system is to be used as the medical imaging device, which creates images on the basis of transit time measurement data on the ultrasonic waves, then for the method it is sufficient for the corresponding raw data, i.e., the transit time measurement data to be captured. In imaging systems based on magnetic resonance, it is also sufficient to capture the raw data of the magnetic resonance tomograph, said data being suitable for conversion to corresponding image data. Consequently, in capturing the at least one image by means of the imaging device, the point is not to produce an actual image in the sense of a photograph or a visual impression, but instead it is sufficient to capture the raw data that forms the basis for the imaging device-specific images and to process the raw data according to the method. The imaging devices used in the method are not limited and comprise the typical imaging devices that are mentioned in the introduction and are used in the medical field.

The actual pose of the manipulator is determined on the basis of the captured image, and a deviation between the target pose and the actual pose is determined. The deviation is a measure of the absolute accuracy. If the repeat accuracy is to be improved, then the target pose may be a pose approached previously and need not be determined by exact coordinates in space.

Calibration parameters are then calculated on the basis of the deviations determined, and are in turn used to calibrate the manipulator. This is typically achieved by adapting a model of the manipulator in a control device of the manipulator, so that commanded control commands result in the exact/accurate approach to the target pose.

To determine the actual pose, the position and orientation of the imaging device relative to the manipulator must be known. To do so, the manipulator may be disposed at a known distance and/or in a known orientation in relation to the imaging device. This can be achieved, for example, by fixedly connecting the manipulator to the imaging device or by fixedly anchoring the manipulator and the imaging device in a known position.

It is likewise possible to provide markers, which are disposed in stationary positions, for example, as described further below. The position and orientation of the manipulator base can also be inferred from certain positions of the end effector. For example, if the manipulator is completely extended (telescoped position), and the end effector of the manipulator and/or the last link of the manipulator is captured, then if the geometry of the manipulator in the telescoped position is known. This makes it possible to increase the accuracy, i.e., calibration, because such positions typically have a very small positional error, i.e., deviations in pose. Furthermore, the position and orientation of the imaging device can be determined relative to the manipulator by so-called referencing or manual guidance of the manipulator. To do so, for example, the end effector is guided manually to a point of known coordinates (reference point). The manipulator can in fact be moved manually for this purpose or controlled manually to the point by a suitable control device. It is also possible to carry out a hand-eye calibration.

In particular, the method may additionally comprise at least the following steps:

Determining a quality parameter indicating the accuracy of the manipulator in approach to the target pose, wherein the quality parameter indicates in particular the absolute accuracy and/or the repeat accuracy of the manipulator; and

Repeating steps a) through f) until the quality parameter has fallen below a predefined quality limit.

Repeating the preceding steps a) through f) increases the accuracy of the calibration because errors that occur can be averaged. In particular, it is possible to approach the target pose from various directions in order to achieve a higher accuracy. Then the method can be terminated when the result falls below a predefined quality parameter, i.e., the desired accuracy has been reached. The accuracy may relate to the repeat accuracy or the absolute accuracy of the manipulator, for example. It is also possible to use other definitions of accuracy to determine the quality parameter or to combine different accuracy values. For example, the quality parameter can be obtained proportionally from a first factor, which determines the absolute accuracy, and from a second factor, which determines the repeat accuracy.

The medical imaging device may preferably be an x-ray imaging device, an ultrasonic imaging device and/or a magnetic resonance imaging device.

These medical imaging devices are typically present in operating rooms or treatment rooms, so that no other traditional measurement systems need be installed or in operation to calibrate the manipulator. Furthermore, use of these medical imaging devices permits calibration of the manipulator with a sufficient accuracy because the subsequent therapeutic or diagnostic procedure is monitored by means of these medical imaging devices. The therapeutic or diagnostic procedure can be monitored with the accuracy provided by the medical imaging device. The same accuracy can then be achieved in calibration of the manipulator so that reliable systems are achieved.

Furthermore, the medical imaging device may be equipped to create two-dimensional and/or three-dimensional images.

If two-dimensional images are created by the medical imaging device or if the device is equipped to do so, then it may be necessary to capture multiple images in order to be able to unambiguously determine the actual pose of the manipulator. However, three-dimensional images have the advantage that a three-dimensional image is usually sufficient to determine the actual pose of the manipulator.

The manipulator and/or the end effector may comprise at least one marker, which is equipped to be captured by the imaging device, and wherein the marker is also captured in capturing the image.

Markers mounted on the manipulator and/or end effector or designed integrally with them have the advantage that they can be designed to be captured reliably by the medical imaging device. If the medical imaging device is an x-ray-based medical imaging device, for example, such as a C arm or a computer tomograph (CT), then the markers may be x-ray markers, which can be clearly identified in the image capture. In particular, the markers may be of a type, such that they produce the fewest possible artifacts in the image created to enable an accurate capture of the markers and to be able to carry out an accurate calibration. In the case of magnetic resonance-based imaging systems, the markers may be fluid-filled objects, in which the fluid is water and/or an alcohol, for example.

The marker may also have a defined geometric shape, which simplifies the determination of the position and/or orientation of the marker on the basis of the captured image.

If the marker has geometrically defined shapes, then the actual pose of the manipulator can be determined quickly and easily. The marker is preferably designed, so that the position and orientation of the marker in space can be determined unambiguously when an image, in particular exactly one image, is compiled.

One example of a suitable shape of the marker is a triangular shape, in which each of the sides is a different length. Other characteristic shapes are also possible. In particular, it is not necessary for the marker to form a physical unit. The marker may consist of a plurality of subunits, which are in a fixed geometric relationship to one another. In the case of an x-ray marker, for example, three individual x-ray markers may be mounted on a corresponding triangular structure or may be mounted on the manipulator in a triangular shape. Other configurations and geometric shapes are also possible. For example, it may be sufficient to arrange two markers on the longitudinal axis of the end effector in the case of end effectors having a rotational symmetry in order to determine its position and orientation in space. An example of a rotationally symmetrical end effector is a drill or a biopsy needle.

In particular, the marker may be designed to be integral with the manipulator and/or the end effector and is preferably designed to be integral with the housing of the manipulator and/or of the end effector. In particular, the manipulator and/or end effector or a part and/or a certain structure of the manipulator and/or end effector may be used as markers.

The integral design of the marker with the manipulator or the end effector in particular makes it possible to save on installation space, so that the marker does not interfere with the diagnostic and/or therapeutic method associated with the calibration method. In particular, the markers may also be equipped to make it possible to differentiate end effectors from one another. For example, a first end effector, which is a drill, may comprise a first marker, and a second end effector, which is a screw-driving tool, for example, may comprise a second marker, so that it is possible in calibration to detect which tool/end effector the manipulator is guiding at the moment.

The marker may also be disposed in a housing of the manipulator and/or of the end effector, wherein the housing of the manipulator and/or the end effector may be translucent and/or transparent for the imaging device.

It is advantageous in particular to mount the markers inside the housing of the end effector/manipulator because this avoids or prevents disturbance by the marker(s). However, it is important to be sure that the housing does not have a negative effect on capture of the markers. Therefore, translucent or transparent housing materials are advantageous. In the case of an x-ray-based imaging device, for example, plastics that are at least partially permeable for x-rays may be used.

The marker may also be releasably connected to the manipulator and/or the end effector, in which case the method may comprise the following step: arranging and/or releasing at least one marker on the manipulator and/or the end effector, with the mounting being accomplished in particular by means of a releasable connection.

If the markers are releasably connected to the manipulator, then it is possible to remove them after calibration to prevent the markers from interfering with carrying out the diagnostic/therapeutic procedure. In this case, the method for calibration may comprise the method steps: disposing at least one marker on the manipulator and/or end effector, so that the marker is preferably also moved together when the manipulator is moved; and detaching the marker from the manipulator and/or end effector after the calibration has been performed. It is not absolutely necessary to detach the marker, but this can be carried out as an optional method step.

In particular steps a) through f) can be carried out for at least two different target positions. If steps a) through f) of the method are carried out for different target positions, then the manipulator can be calibrated in the entire working range of the manipulator, and a high absolute accuracy can be achieved throughout the entire working range. Various poses that are distributed over the entire working range of the manipulator are typically used as target poses. The quality parameter, below which the measured value may optionally fall, can also be defined in different ways for each individual target pose. In particular, a position-dependent quality parameter may be defined. Thus, for example, the manipulator may have a very high accuracy when used in a medical center, which typically corresponds to a surgical environment, whereas a lower accuracy is sufficient in the peripheral area of the working range of the manipulator.

In particular, the manipulator can be moved when capturing an image of at least one part of the manipulator and/or at least one part of end effector of the manipulator by means of the medical imaging device and need not be stationary. In this case, the image is captured at the point in time when the manipulator reaches the target pose.

If the calibration is carried out not on the stationary manipulator but instead on a moving manipulator, as described above, it is also possible to improve the accuracy in dynamic running of paths. The deviation of a plurality of target poses in succession from the actual poses is determined in this process. The captured images may be images which are aligned in a row and are processed like a video. Here again, it does not matter that the captured images are in fact reproduced as visual impressions, but instead the issue is only that the raw data of the images is processed further.

The at least one calibration parameter can additionally depend on the velocity or acceleration of the manipulator in approach to the target position. If the calibration parameter also depends on influencing variables, such as velocity and acceleration of the manipulator, in addition to depending on just the offset, i.e., the geometric deviation, then the achievable accuracy can be further increased because inertia effects are also taken into account in calibration. It is also possible to take other influencing factors into account in determining the calibration parameter(s). For example, the temperature of the manipulator or of part of the manipulator may be taken into account. If the typical wear characteristics of the manipulator are known, then the calibration parameter may also be a function of time and may be adapted to the duration of operation of the manipulator, for example.

The quality parameter can be updated continuously during operation of the manipulator, and a warning can be output when the quality parameter exceeds the predefined quality limit. If the quality parameter is updated continuously during operation of the manipulator, then it is possible to ensure that the desired accuracy is maintained with sufficient accuracy. If the quality parameter is exceeded, a warning can be output, prompting the user to perform a new calibration. In addition, the manipulator system can be stopped after detecting that the quality parameter has been exceeded or the manipulator system may be controlled at a reduced velocity in a safety mode.

Furthermore, the calibration parameter(s) may be an input variable for a computer model. Based on the computer model, the pose with which the manipulator is charged can then be corrected to achieve a higher absolute accuracy, for example. In a simple illustrative example, the computer model takes into account, for example, offset values of the individual axes of the manipulator as calibration parameters. In a more complex computer model, for example, statistical deviations in six degrees of freedom can be determined for each axis of the manipulator. Likewise, dynamic deviations, which depend on the velocity and/or acceleration of the respective axis or axes of the manipulator may also be taken into account. The influence of external forces or temperature can also be included in the computer model. To do so, the manipulator system may include additional sensors, preferably force sensors and/or torque sensors and/or temperature sensors. In addition, a load carried by the manipulator may also be taken into account in the computer model. This can be done by taking into account such parameters as the weight of the load, the center of gravity of the load and/or the like.

The manipulator may be a mobile manipulator, which has a mobile platform, wherein a marker, which is equipped to be captured by the imaging device is preferably arranged on the mobile platform, and wherein the marker is also captured in capturing the image.

Mobile manipulators have the advantage that they can be used flexibly in different locations. In other words, the actual manipulator is arranged on a mobile platform, which can move (freely) in space. This is advantageous in particular in tight spaces, where the manipulator acts together with humans because the manipulator can be positioned freely and thus the best possible access to the work area can be granted to the human and/or the manipulator.

The mobile manipulator may comprise at least one coupling means, wherein the mobile manipulator can be secured in a stationary position by using the coupling means. To be able to accurately position and orient the mobile manipulator in relation to the imaging device in order to perform an accurate calibration, the manipulator may comprise coupling means, with which it can be secured in a stationary position. For example, the coupling means may be a mechanical coupling means, with which the manipulator can be secured on a stationary object. The coupling means may then be designed, for example, as a projection and a complementary stationary coupling means may be designed as a corresponding setback, for example, with a conical shape, such that the cone fits into a mating cone in a form-fitting manner. Other geometric shapes are also possible. In particular, the coupling means may be locked to one another. This locking may be accomplished by means of a form-fitting and/or force-locking connection. For example, the coupling means may be equipped with magnets, so that they snap together when the coupling means are coupled. Other coupling means comprising locking levers, snaps or the like are also conceivable.

The imaging device may comprise a complementary coupling means, which can be coupled to the coupling means of the mobile manipulator in order to secure the mobile manipulator in relation to the imaging device. If the imaging device is equipped with a complementary coupling means, then there may be a direct coupling of the mobile manipulator and the imaging device, so that the accuracy in calibration can be further increased because the imaging device and the manipulator are aligned accurately with one another. In particular, it should be pointed out that the coupling means can be releasably connected to one another and are preferably quick coupling means, i.e., they can be coupled to one another and/or uncoupled from one another without the use of tools.

In addition, the manipulator system may also comprise at least one stationary marker, which is equipped to be captured by the imaging device, such that the marker is also captured when the image is captured. Stationary markers, such as the markers described above, which are arranged on the manipulator, make it possible to establish a spatial reference (position and/or orientation) between the end effector and/or the manipulator and the stationary coordinate system. A calibration can be carried out in this way. In particular, the markers may also be arranged on the manipulator base of the manipulator and/or of the mobile manipulator.

This object is additionally achieved by a control device, which comprises at least one processor and one data memory, wherein the control device is equipped to control at least one manipulator according to the method described above. The control device may control at least one manipulator and/or one mobile manipulator according to the method described above. It is also possible to control a plurality of manipulators by means of one control device. For example, a plurality of manipulators, which are jointed arm manipulators, can be controlled by a control device and calibrated according to the method.

This object is additionally achieved by a manipulator system comprising at least one manipulator and one medical imaging device as well as a control device, wherein the control device is equipped to control at least one manipulator according to the method described above. Such manipulator systems are typically used in treatment rooms and operating rooms and make it possible to calibrate the manipulator without providing or installing additional measurement systems, so that the costs in calibration can be reduced substantially. In particular, the calibration can be performed more frequently because all the required systems, such as the control device and the imaging device, are available on site.

This object is additionally achieved by a computer-readable medium, comprising program commands that prompt a control device described above to execute the method described above.

### BRIEF DESCRIPTION OF THE DRAWINGS

The accompanying drawings, which are incorporated in and constitute a part of this specification, illustrate exemplary embodiments of the invention and, together with a general description of the invention given above, and the detailed description given below, serve to explain the principles of the invention.

FIG. 1 shows a schematic diagram of a manipulator system;

FIG. 2 shows a schematic diagram of an additional manipulator system;

FIG. 3 shows another diagram of yet another manipulator system; and

FIG. 4 shows a schematic diagram of a method for calibration.

### DETAILED DESCRIPTION

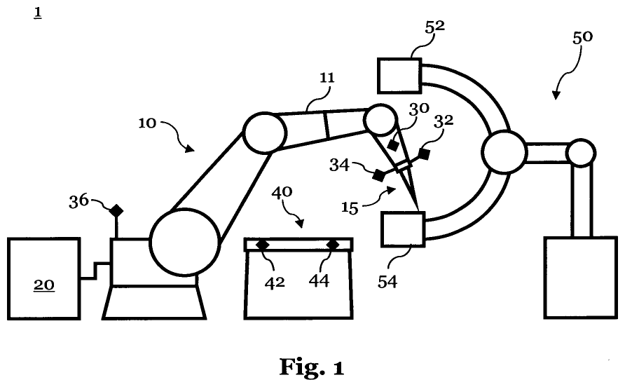

FIG. 1 shows in particular a manipulator system 1, comprising a manipulator 10, which is equipped to carry out diagnostic and/or therapeutic procedures. The manipulator 10 comprises a housing 11 and guides an end effector 15, which is diagramed schematically in FIG. 1 as a biopsy needle. Other end effectors may also be guided by means of the manipulator 10.

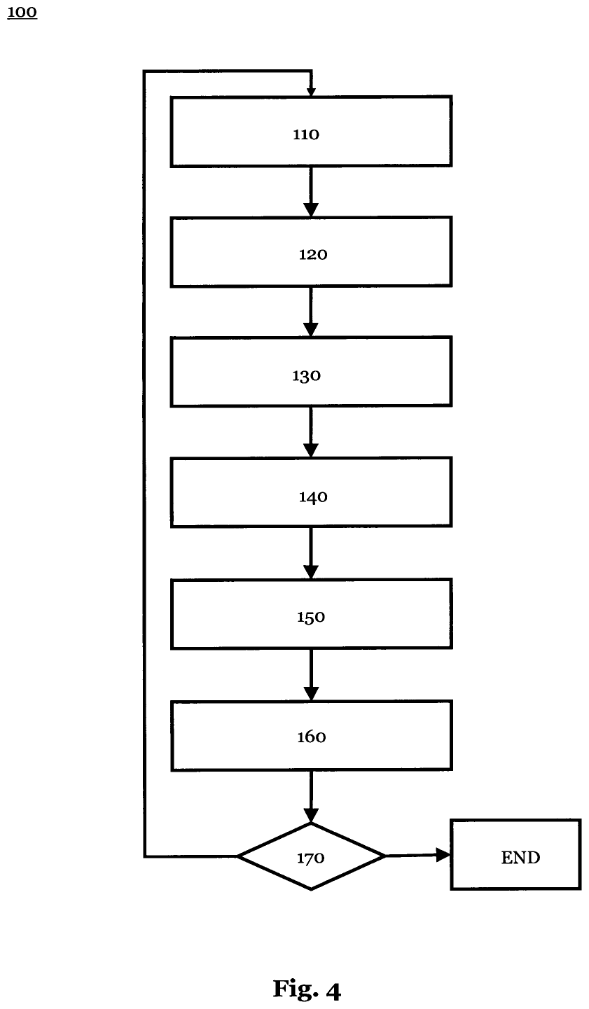

The manipulator is controlled by the control device 20, which may comprise both software and hardware components. In particular, the control device is equipped to control the manipulator in accordance with the method described below (cf. FIG. 4). The manipulator additionally comprises markers 32, 30, 34, which are mounted on a last link and/or on the end effector. In particular, the markers 32, 30, 34 may be arranged releasably.

The markers 32, 30, 34 are designed to be captured by the medical imaging device 50. The medical imaging device 50 may be any medical imaging device, which is used in the medical field. In the example shown here, it is designed as a C arm. The C arm has an x-ray source 52 and an x-ray recording unit 54. The x-ray recording unit 54 captures the x-rays emitted by the x-ray source 52 to capture an image.

In addition, the manipulator system may comprise a patient bed 40, which may be equipped with stationary markers 42, 44. A marker 36 may be associated with the manipulator 10 at its manipulator base. The manipulator system 1, which can be seen in FIG. 1, preferably comprises a stationary manipulator 10 and a stationary imaging device 50, i.e., these are secured in space relative to one another.

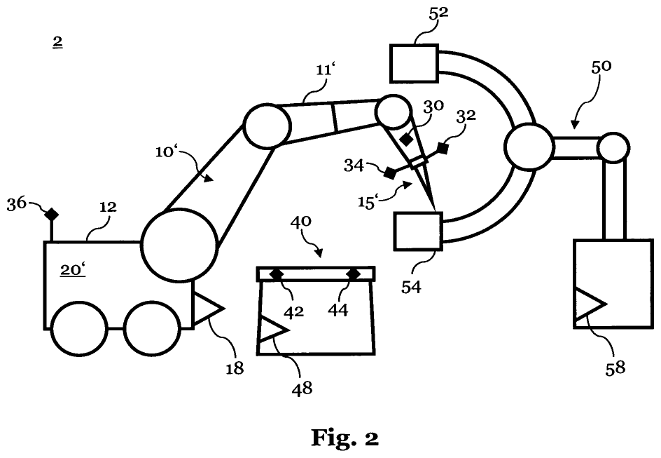

FIG. 2 shows another manipulator system 2, which can be used for diagnostic and/or therapeutic procedures. Instead of the manipulator 10 from FIG. 1, a mobile manipulator 10′ is used here. The mobile manipulator 10′ comprises a housing 11′ and end effector 15′ and a mobile platform 12, by means of which the mobile manipulator 10′ can move freely in space. A control device 20′, which is equipped to control the manipulator and to execute the method described here (cf. FIG. 4), is associated with the manipulator 10′.

The manipulator additionally comprises the markers 30, 32, 34, 36 described above. The manipulator system additionally comprises an imaging device 50 (C arm), which has an x-ray source 52 and an x-ray recording unit 54. In addition, a patient bed 40 having markers 42, 44 may be associated with the manipulator system 2. The mobile manipulator 10′ comprises coupling means 18, which can couple in a form-fitting manner with the complementary coupling means 58 and/or 48 of the imaging device 50 and/or of the patient bed 40, for example. The manipulator can therefore be secured in relation to the patient bed 40 and/or the imaging device 50, so that the calibration of the manipulator 10 is simplified.

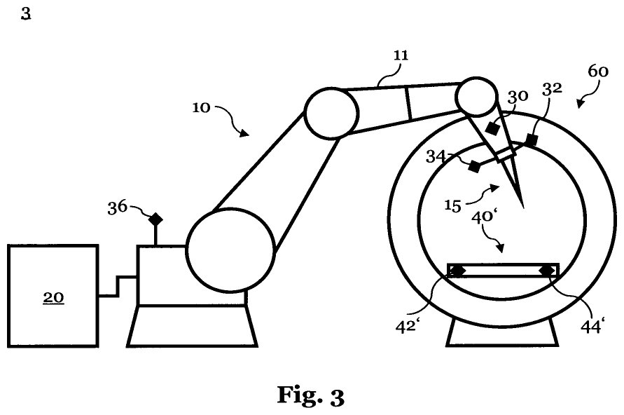

FIG. 3 shows another manipulator system 3, which can be used for diagnostic and/or therapeutic procedures. In addition to the manipulator 10 already described above (cf. description of FIG. 1), the manipulator system 3 comprises an imaging device 60, which may be a computer tomograph or a magnetic resonance tomograph, for example. A patient bed 40′ comprising the corresponding markers 42′, 44′ is arranged in the imaging device 60. The same reference numerals used in FIGS. 1 to 3 also refer to the same components of the manipulator systems. In particular, the components in the individual manipulator systems 1, 2 and 3 can be exchanged. Thus, for example, a mobile manipulator 10′ may also be used together with the imaging unit 60.

FIG. 4 shows a method 100 for calibrating a manipulator 10, 10′ of a diagnostic and/or therapeutic manipulator system 1, 2, 3, wherein the manipulator system comprises a medical imaging device 50, 60. In a first method step 110, at least one target pose is approached by means of the manipulator. The pose here refers to the position and orientation of the manipulator in space. If the target pose is approached from different positions, the method must be carried out several times.

In a second method step 120, at least one image of a part of the manipulator and/or at least one part of the end effector of the manipulator is captured by means of the medical imaging device when the manipulator has approached the target pose or has just passed through it. In doing so, it is not necessary to generate an image in the sense of a photograph or an image that can be displayed visually; it is instead sufficient for the imaging device to capture raw data suitable for being processed further to yield a typical medical diagnostic image.

In the third method step 130, the actual pose of the manipulator is determined by means of the captured image and/or the captured raw data.

Finally, the deviation between the target pose and the actual pose of the manipulator is determined in method step 140, and in step 150, a calibration parameter is ascertained, based on this deviation thereby determined. The calibration parameter may be based on purely geometric parameters and/or additional parameters, such as the velocity of approach or the acceleration of the manipulator, the heating of the manipulator, the running time of the manipulator and the like.

Finally, the manipulator is calibrated in method step 160. In method step 170, it is possible to verify whether the manipulator has already achieved the desired accuracy, i.e., whether a quality parameter has fallen below a predefined quality limit. If this is the case, the method can be terminated. If this is not yet the case, the method must be carried out again. In particular, the method can be carried out for a plurality of target poses.

While the present invention has been illustrated by a description of various embodiments, and while these embodiments have been described in considerable detail, it is not intended to restrict or in any way limit the scope of the appended claims to such detail. The various features shown and described herein may be used alone or in any combination. Additional advantages and modifications will readily appear to those skilled in the art. The invention in its broader aspects is therefore not limited to the specific details, representative apparatus and method, and illustrative example shown and described. Accordingly, departures may be made from such details without departing from the spirit and scope of the general inventive concept.

### LIST OF REFERENCE NUMERALS

1, 2, 3 Manipulator system
10 Manipulator
11 Housing
12 Mobile platform
15 End effector
10′ Mobile manipulator
11′ Housing of mobile manipulator
15′ End effector of mobile manipulator
20 Control device
30, 32, 34, 36 Marker of manipulator
18 Coupling means
40 Patient bed
40′ Patient bed
42, 42′, 44, 44′ Marker of patient bed
50 Imaging device (C arm)
52 X-ray source
54 X-ray capture unit
58 Complementary coupling means
48 Complementary coupling means
60 Imaging device (MRI)
100 Method
110, 120, 130, 140, 150, 160, 170 Method steps

What is claimed is:

1-20. (canceled)

21. A method for calibrating a robotic manipulator of a diagnostic and/or therapeutic manipulator system, wherein the robotic manipulator system includes at least one medical imaging device, the method comprising:
a) approaching at least one target pose with the robotic manipulator;
b) capturing at least one image of at least one part of the robotic manipulator and/or at least one part of an end effector of the robotic manipulator with the medical imaging device when the robotic manipulator has moved to the target pose;
c) determining the actual pose of the manipulator using the captured image;
d) determining a deviation between the target pose and the actual pose of the robotic manipulator;
e) calculating at least one calibration parameter based on the determined deviation; and
f) calibrating the robotic manipulator based on the at least one calibration parameter.

22. The method of claim 21, further comprising:
determining a quality parameter indicative of the accuracy of the manipulator in approach to the target pose, wherein the quality parameter indicates at least one of the absolute accuracy or the repeat accuracy of the robotic manipulator; and
repeating steps a) through f) until the quality parameter has dropped below a predefined quality limit.

23. The method of claim 21, wherein the medical imaging device is at least one of an X-ray imaging device, an ultrasonic imaging device, a positron emission tomography imaging device, or a magnetic resonance imaging device.

24. The method of claim 21, wherein the medical imaging device is configured to prepare at least one of two-dimensional or three-dimensional images.

25. The method of claim 21, wherein:
at least one of the robotic manipulator or the end effector comprises at least one first marker that is configured to be captured by the imaging device; and
capturing at least one image comprises capturing the at least one first marker in the image.

26. The method of claim 25, wherein the first marker has a defined geometric shape that enables the determination of at least one of the position or orientation of the first marker on the basis of the captured image.

27. The method of claim 25, wherein the first marker is integral with the robotic manipulator or the end effector.

28. The method of claim 25, wherein:
the at least one first marker is arranged in a housing of the manipulator or of the end effector; and
the housing of at least one of the robotic manipulator or of the end effector is translucent or transparent for the imaging device.

29. The method of claim 25, wherein the first marker is releasably connected to at least one of the robotic manipulator or to the end effector.

30. The method of claim 21, wherein steps a) through f) are carried out for at least two different target poses.

31. The method of claim 21, wherein:
the manipulator does not stand still during capture of an image of at least one part of the robotic manipulator or at least one part of the end effector with the medical imaging device; and
the image is captured when the robotic manipulator has reached the target pose.

32. The method of claim 21, wherein the at least one calibration parameter is a function of at least one of a velocity or an acceleration of the robotic manipulator in approach to the target pose.

33. The method of claim 22, further comprising:
updating the quality parameter during operation of the robotic manipulator; and
outputting a warning when the quality parameter exceeds the predefined quality limit.

34. The method of claim 21, wherein:
the robotic manipulator is a mobile robotic manipulator comprising a mobile platform with at least one second marker configured to be captured by the medical imaging device; and
capturing the at least one image comprises capturing the at least one second marker in the image.

35. The method of claim 34, wherein:
the mobile robotic manipulator further comprises at least one first coupling means; and
the method further comprises securing the mobile robotic manipulator in a stationary position with the first coupling means.

36. The method of claim 34, wherein:
the medical imaging device comprises a second coupling means complementary to the first coupling means and configured to be coupled with the first coupling means; and
the method further comprises securing the mobile robotic manipulator relative to the medical imaging device by coupling the first and second coupling means.

37. The method of claim 21, wherein:
the manipulator system comprises at least one stationary third marker configured to be captured by the medical imaging device; and
capturing the at least one image comprises capturing the at least one third marker in the image.

38. A control device comprising at least one processor and one data memory, wherein the control device is configured to control at least one robotic manipulator according to the method of claim 21.

39. A diagnostic and/or therapeutic manipulator system, comprising:
at least one robotic manipulator;
at least one medical imaging device; and
a control device, wherein the control device is configured to control the at least one robotic manipulator according to the method of claim 21.

40. A computer program product, comprising program code stored on a non-transitory, computer-readable medium, the program code configured to, when executed by a computer, cause the computer to:
a) approach at least one target pose with a robotic manipulator;
b) capture at least one image of at least one part of the robotic manipulator and/or at least one part of an end effector of the robotic manipulator with a medical imaging device when the robotic manipulator has moved to the target pose;
c) determine the actual pose of the manipulator using the captured image;
d) determine a deviation between the target pose and the actual pose of the robotic manipulator;
e) calculate at least one calibration parameter based on the determined deviation; and
f) calibrate the robotic manipulator based on the at least one calibration parameter.
